(19) United States
(12) Patent Application Publication
(10) Pub. No.: US 2022/0361722 A1
(43) Pub. Date: Nov. 17, 2022

(54) METHOD AND APPARATUS FOR DISPENSING TOILET PAPER

(71) Applicant: Samantha Jane Soltau, Venice, CA (US)

(72) Inventor: Samantha Jane Soltau, Venice, CA (US)

(21) Appl. No.: 17/742,794

(22) Filed: May 12, 2022

Related U.S. Application Data
(60) Provisional application No. 63/187,514, filed on May 12, 2021.

(51) Int. Cl. A47K 10/38; A61L 2/10; H05B 47/115; H05B 47/16

(52) U.S. Cl. CPC A47K 10/38; A61L 2/10; H05B 47/115; H05B 47/16; A47K 2010/389

(57) ABSTRACT

A housing is provided to enclose a heating device configured to warm toilet paper and irradiate the toilet paper with germicidal UV light. An additional light may be provided for the convenience of the user at night or other low light applications. The devices may be initiated by the user, initiated by a motion or touch sensor, or initiated by a timer. A smart device may be utilized to provide user configured preferences. The devices may be powered by a battery pack or may receive power from a wall outlet.

### CROSS-REFERENCE TO RELATED APPLICATIONS

This application claims the benefit of U.S. Provisional Patent Application No. 63/187,514, filed May 12, 2021. The disclosure of the priority application in its entirety is hereby incorporated by reference into the presence application.

### FIELD OF THE INVENTION

This invention relates generally to dispensing a web material; and more particularly to a method and apparatus for dispensing toilet paper in a sanitary, ergonomic and convenient fashion; and more particularly still to a toilet paper roll dispenser that includes U.V. germicidal light, a heat source to warm the toilet paper and a courtesy light.

### BACKGROUND

There are many types and styles of toilet paper dispensers. For example, there are single spindle arm holders, spring loaded and pivoting spindle arm holders, locking holders, recessed holders and commercial holders for single and double rolls, among others. In general, each type of holder includes a spindle which extends through the roll of toilet paper such that the toilet paper is rotatably positioned on the spindle.

The toilet paper itself generally is manufactured using paper in sheets or on a roll for wiping oneself clean after urination or defecation. More specifically, it is generally a tissue paper product typically used to clean the anus and surrounding area of feces after defecation and/or to clean the perineal area of urine after urination or other bodily fluid. In addition to cleaning and absorbing feces and urine, the toilet paper acts as a layer of protection for the hands during these processes. Usually, it is supplied as a long strip of tissue paper wrapped around a paperboard core in a dispenser near a toilet. The resulting roll of web materials may be referred to by a variety of names including toilet paper, toilet tissue, wipes, toilet roll and/or loo roll (referred to collectively herein as “toilet paper” or “toilet tissue”).

One toilet paper holder is described in U.S. Pat. No. 5,697,577. In that patent the toilet paper is premoistened and the toilet paper is located in a chamber that is substantially sealed to prevent evaporation of the moisture on the toilet paper roll. A door is provided to selectively provide access to the chamber and to the moistened toilet paper. Heating the moistened toilet paper is accomplished through wires attached to the inside of the housing or a light bulb located below the toilet paper. A button is located on the front of the housing to initiate heating. In either case, however, due to the methods of heating, there is a significant drain of power and time associated with warming the outer-most layer of toilet paper.

Another toilet paper holder is described in U.S. Pat. No. 6,476,365. In this holder, toilet paper with an anti-bacterial lotion is heated by using a heating element positioned in the bottom portion of the housing. The housing pivots to an open position to provide access to the toilet paper. The heating element is not described.

Therefore, there is a need in the art for a method and apparatus for providing a toilet paper dispenser with improved sanitary, ergonomic and convenience features. The present invention addresses these and other needs in the art.

### SUMMARY

A preferred embodiment of a method and apparatus constructed in accordance with the principles of the present invention is a device that provides additional comfort and a more sanitary environment when using toilet paper. More specifically, the device includes various features and options to warm the toilet paper and irradiate the toilet paper with germicidal UV light. Further, an additional light may be provided for the convenience of the user at night or other low light applications. Each of the foregoing may be initiated by the user, initiated by a motion or touch sensor, and/or initiated by a timer. To further increase flexibility, the device may be powered by a battery pack or may be plugged into a wall outlet.

The device includes optional housings to locate the toilet paper within the housing, on a face of the housing, adjacent to or above an existing toilet paper holder. Thus, the device may be installed as an initial fixture within a bathroom (e.g., a bathroom being constructed or remodeled) or as a retrofit addition for those bathrooms already having fixtures.

One advantage of the device is that the device uses a heating methodology for the toilet paper that quickly warms the several outermost layers. It does this by using a heater element with a fan blowing across the heater element. Using this type of convection heat in close proximity to the toilet paper warms the outermost layers of the toilet paper rapidly. Another type of heating that may be used is a polyimide heater film. Such films provide fast preheating speed, are durable, lightweight and may include an adhesive to provide specific desired placement of the heating element.

Another advantage of the device is that use of a germicidal UV light reduces the risk of transmission of germs between users of the toilet paper and toilet paper dispenser.

Still another advantage of the device is that the device can include electronic controls, thereby building in a variety of optional control functions. The control functions can be preprogrammed or can be provided as options for individual users. An optional feature of the device is use of an app (or application program), which may be operated on a smart device to control the toilet paper dispenser functions. More specifically, various functions may be performed and user preferences established—such as whether to use each function, how long and when to turn on the UV lights, how long and when to turn on the heater (as well as the preferred temperature), how long and when to turn on the low light level courtesy light and whether to initiate the functions by motion, touch or time, among others.

Therefore, according to one aspect of the invention, there is provided: an apparatus for dispensing toilet tissue, comprising: a housing; a heating element located on or within the housing and proximate the toilet tissue; a germicidal lamp within the housing, and arranged and configured to irradiate the toilet tissue; and a control device operatively connected to the heating element and the germicidal lamp, the control device enabling operation of the heating element and the germicidal lamp.

Further in accordance with some embodiments, the aspect of the invention described in the previous paragraph further includes one or more of the following additional features: wherein the toilet tissue is rotatably mounted within at least a portion of the housing and the housing includes a first void through which the toilet tissue is dispensed; wherein the housing is located above an existing toilet tissue holder, the housing including one or more second voids within a bottom portion of the housing, the heating element includes a fan to direct heated air through the voids and onto the toilet tissue; wherein the control device includes a timer for the heating element and/or the control device includes a timer for the germicidal lamp; wherein the control device includes a microprocessor and memory, the microprocessor being programmable to enable individual user preferences on duration and temperature of the toilet tissue heating and to store the individual preferences in the memory location; further comprising a motion sensor, the motion sensor initiating operation operatively connected to the control switches and arranged and configured to initiate at least one of the heating element and the germicidal lamp; wherein the motion sensor is located on the top of the housing, whereby a user motion triggers the motion sensor with a gesture above the housing; wherein the motion sensor is located on the bottom of the housing, whereby a user motion triggers the motion sensor with a gesture below the housing; and further comprising a courtesy light.

In accordance with still further embodiments, the aspect of the invention described above may further include wherein the control device is arranged and configured to communicate with a smart device and to receive user selected preferences from the smart device related to at least one of the heating element, the germicidal lamp, and the courtesy light. In other embodiments, the heating element is a polyimide heater film. And in still further embodiments, the heating element is mounted on an external surface of the housing.

According to another aspect of the invention, there is provided: a method of dispensing toilet tissue in an ergonomic manner, comprising: transmitting heat to at least the outer layers of a roll of toilet tissue from a heating element located on or within a housing; shining a germicidal lamp on the outer layers of a roll of toilet tissue from a light source located within the housing; and providing a control device, the control device operatively connected to the heating element and the germicidal lamp and enabling a user to selectively operate the heating element and the germicidal lamp.

While the invention will be described with respect to preferred embodiment configurations and with respect to particular components and structures, it will be understood that the invention is not to be construed as limited in any manner by either such components and structures described herein. Instead, the principles of this invention extend to any method and apparatus in accordance with the claims.

These and other variations of the invention will become apparent to those skilled in the art upon a more detailed description of the invention. The advantages and features which characterize the invention are pointed out with particularity in the claims annexed hereto and forming a part hereof. For a better understanding of the invention, however, reference should be had to the drawings which form a part hereof and to the accompanying descriptive matter, in which there is illustrated and described a preferred embodiment of the invention.

### BRIEF DESCRIPTION OF THE DRAWINGS

Referring to the drawings, wherein like numerals represent like parts throughout the several views:

FIG. 1 is a schematic functional block diagram of the several devices which may be included in some first embodiments constructed in accordance with the principles of the present invention.

FIG. 2 is a schematic functional block diagram of the several devices which may be included in some second embodiments constructed in accordance with the principles of the present invention.

FIG. 3a is a logical flow diagram of a method in accordance with embodiments of the present invention.

FIG. 3b is an alternative embodiment logical flow diagram of a method in accordance with embodiments of the present invention.

FIG. 4 is a perspective view of a housing which may be utilized in connection with the first embodiment of the present invention.

FIG. 5a is a schematic of a 12 volt DC circuit that may be utilized in connection with embodiments of the present invention.

FIG. 5b is a schematic of a 24 volt DC circuit that may be utilized in connection with embodiments of the present invention.

FIG. 6 is a schematic of a smart device 202 utilizing an application of stored programming steps to control various functions of embodiments of the present invention.

FIG. 7 is a first preferred embodiment housing which may be utilized with embodiments of the present invention.

FIG. 8 is a second preferred embodiment housing which may be utilized with embodiments of the present invention.

### DETAILED DESCRIPTION

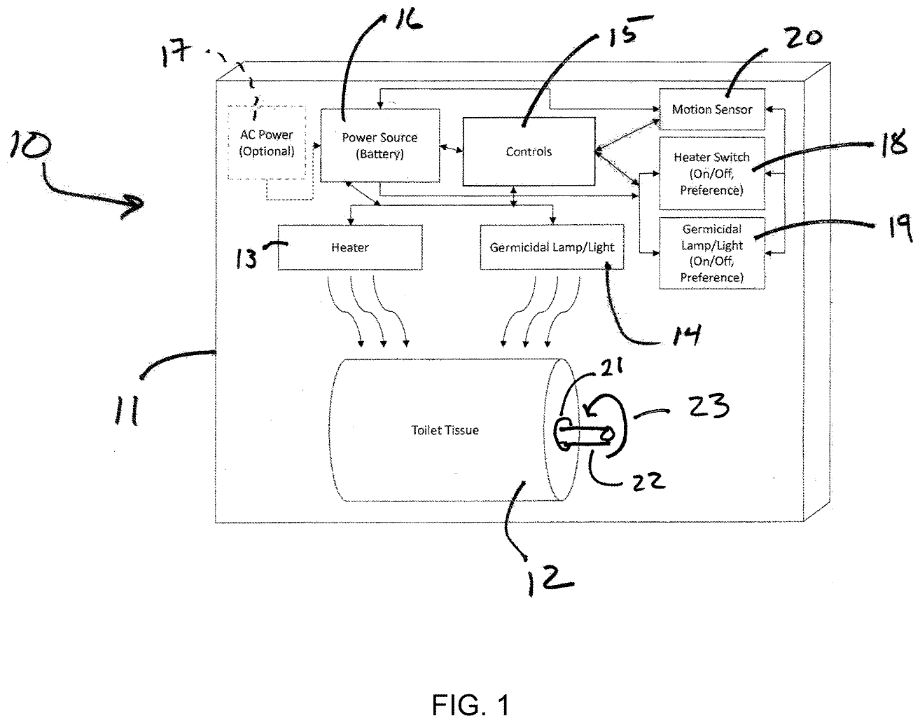

The principles of the present invention apply particularly well to its application as a toilet paper dispenser. However, devices constructed in accordance with the principles of the present invention might be used in other dispenser environments for a tissue, wipe or other web material. Prior to describing the individual components of the dispenser in detail, an overview of the dispenser will be provided with reference to FIGS. 1-4.

Turning first to FIG. 1, an example of a dispenser device constructed in accordance with the principles of the present invention is shown generally at the designation 10. The dispenser 10 includes a housing 11 having a front, back, top and two sides. In some embodiments, the bottom of the housing is open to enable dispensing of the toilet paper. However, in other embodiments, the bottom may be partially closed. In the latter case, vents or louvres may be provided for the heat and UV light to operatively reach the toilet paper 12 roll located beneath the dispenser 10. In yet other embodiments, the roll of toilet paper 12 is mounted on the front of the housing 11.

In the embodiment illustrated in FIG. 1, the toilet tissue 12 resides within the housing 11, is wound on a tube 21, with the tube rotatably mounted on a spindle 22, wherein rotation of the roll of toilet tissue 12 about the tube (shown at 23) dispenses continuous connected sheets of toilet tissue 12 from the roll. A heater 13 and a germicidal lamp 14 are located near the toilet tissue 12. The heater 13 and germicidal lamp 14 are connected to controls 15 and power source. The power source can be a battery 16 or optionally may include AC power (as shown in broken line with the designation 17). Controls 15 can be simple on/off switches, may include appropriate circuitry to provide timers and other features, and/or may include an application for programming control of the dispenser 10 using a smart device 202 (described further below).

A motion sensor 20 may be provided to turn the heater 13 and/or the germicidal lamp 14 on when the dispenser 10 is approached. The motion sensor 20 may be activated by specific movement(s). For example, a foot movement below the housing 11 or a hand movement above, in front of, or by the side of the housing, may be used, among others. Heater switch 18 and germicidal lamp switch 19 also provide user control and are connected to control block 15.

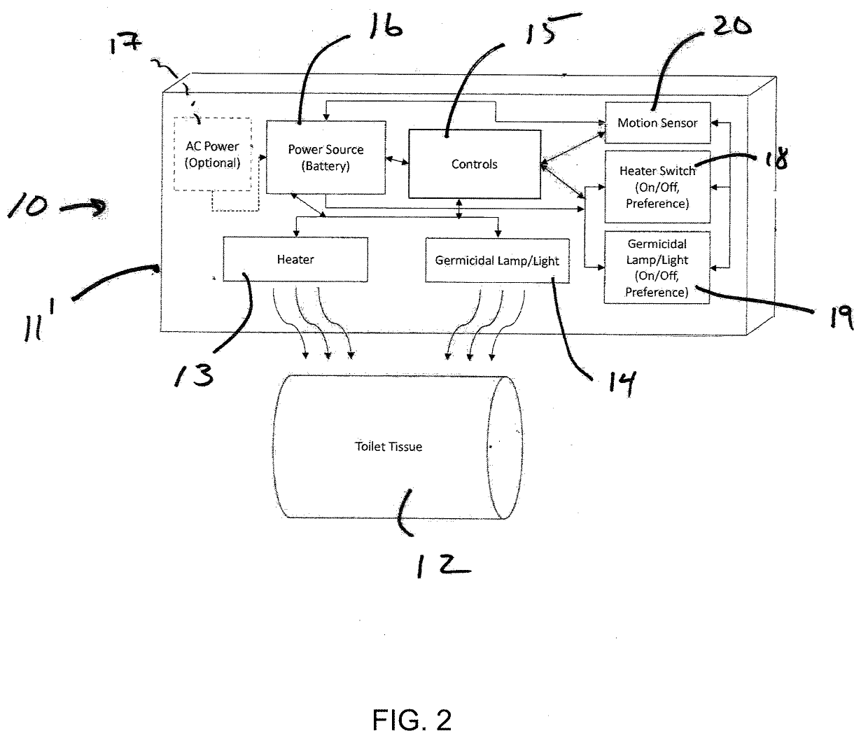

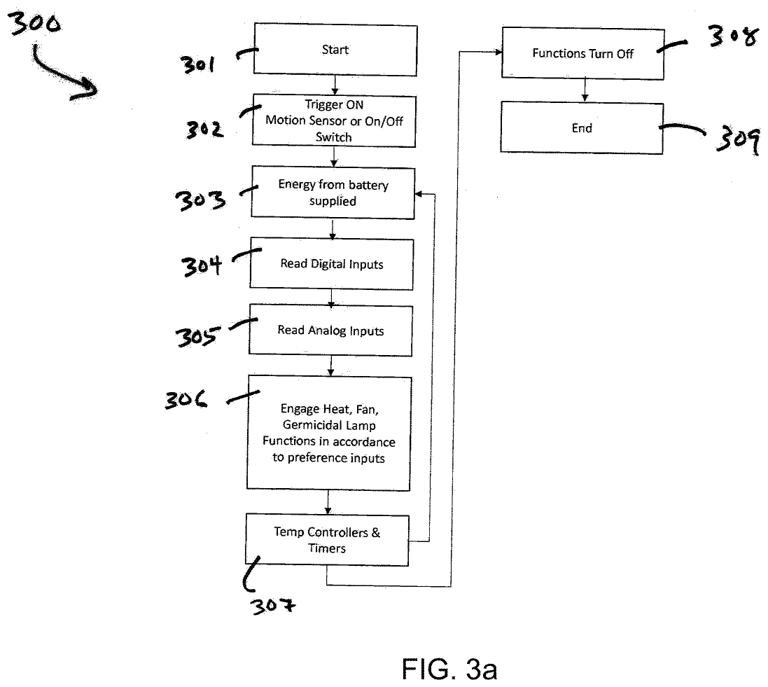

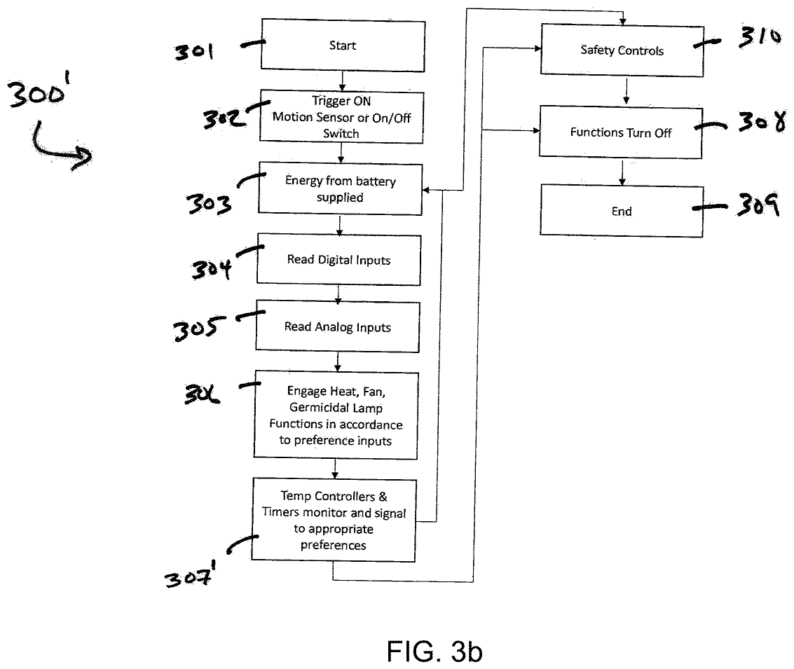

Turning to FIG. 2, an alternative embodiment dispenser 10′ is illustrated. In this embodiment the toilet tissue 12 is located below the housing 11′. However, the other devices and controls are the same as described above in connection with FIG. 1. FIGS. 3a and 3b illustrate the method employed by the dispenser 10 (discussed in more detail below).

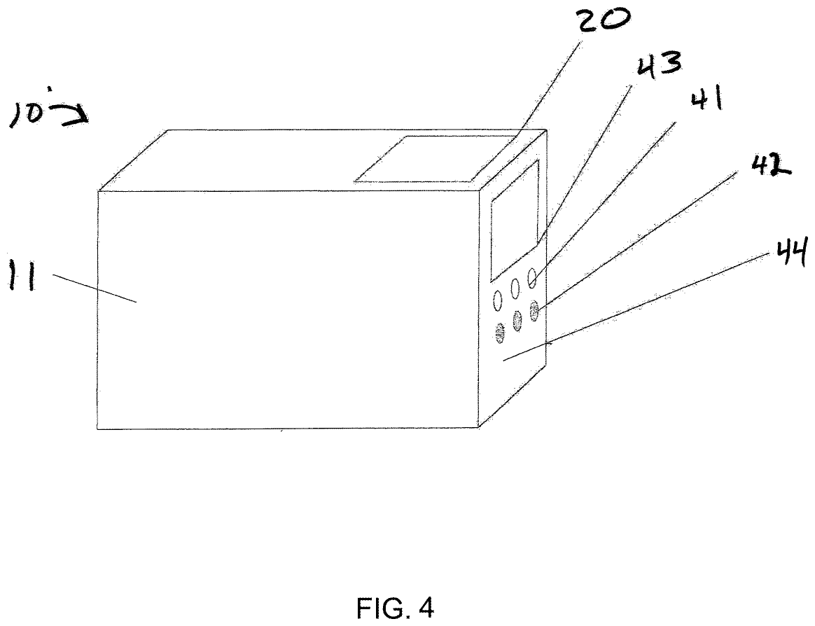

FIG. 4 is a perspective view of an embodiment of the housing 11. Included on the exterior of the housing are motion sensor 20 and access door 43. Lights 41 are provided to indicate the status of the various devices within the housing 11 and switches 42 are provided for a user to control the various functions of the dispenser 10. The switches 42 cooperatively interact with the heater switch 18 and germicide lamp 19 and are operatively connected to the controls 15. It will be appreciated that while the access door 43, lights 41 and switches 42 are illustrated as being located on housing side panel 44, such elements may be located on the other surfaces of the housing 11.

### Heater 13

The heater 13 warms the toilet paper 12 to a temperature higher than the ambient temperature. Preferably, the time for the toilet paper 12 to be heated to the designated temperature is almost immediate (e.g., within several seconds) or may be based on pre-planned usage. In order to preserve battery life, preferably the heat function is not continuous. However, use of the optional AC power 17 enables more continuous operation and for use in commercial or other high use environments.

The heater 13 may be based on a number of different types of heating. A preferred embodiment uses heat transfer by thermal convection or radiation but the heater 13 may use conduction methods as well. In addition to the heat generation element, a fan mechanism may be provided to concentrate and direct heated air to the target area (e.g., the toilet paper 12). Internal structural elements within the housing 11 may be included to further focus heated air to the outer plies of the toilet paper 12. Depending on the housing material, a heat shield may be utilized to protect the housing 11 and shield the housing 11 from becoming hot to a user's touch.

In one embodiment the heater 13 uses convection heat where the heater 13 comprises a coil energized by the battery 16 (or other source). Preferably, the toilet paper 12 is heated to between 100 degrees F. to 120 degrees F. (i.e., corresponding to 25-50 degrees F. above a typical ambient temperature). The heater 13 heats the air surrounding the coil and the air is then directed to heat the targeted toilet tissue 12. In another embodiment, the heating component utilizes conduction, with a mode using infrared heating. Here the heat generating components are targeted at the toilet tissue 12. Targeted air may also be used in this embodiment to spread heat more evenly at the intended target. In one embodiment, use of targeted air may function to rotate the toilet paper 12 in order to more uniformly heat the toilet tissue 12 and/or to dispense the same to a user. The timing and air trajectory may be adjusted in order to dispense an optimal amount.

In yet another embodiment, the heater 13 may include a polyimide heater film. It will be appreciated that such heater film includes a polyimide as an outer insulator and a thermos foil as an inner electric heating element. The insulator and heating elements are then sealed using high temperature and high pressure. Advantages of using polyimide film include it being thin, light weight and flexible. This type of heater may operate at 12V DC and include an adhesive film on one side to locate and place the film at a desired location. Such heaters are commercially available, for example under model designation 988704 and manufactured by Icstation of Shenzhen, China.

It will be appreciated by those of skill in the art that sensors and limits may be employed to communicate with the various controls. For example, timers, thermal controls, temperature testing, etc. may be used. More specifically, in the event that various types of devices are utilized as part of the heater 13, a temperature sensor and/or a controller device may be employed to control the heat output.

### Germicidal Lamp 14

The germicidal lamp 13 is intended to sterilize the toilet paper 12 and may comprise an ultraviolet C (UVC) light or lights. In some embodiments, multiple lights may be employed to better target the circumference of the toilet paper 12 roll. The germicidal lamp(s) 13 is powered by the battery 16 (or optional AC power 17). It will be appreciated that the germicidal lamp 13 could be either a UV tube lamp(s), UV bulb(s), UB Diode(s), or other UV light sources.

### Battery 16

Battery 16 is a preferred power source for the dispenser 10 and is sized and configured to supply power to the various components within the housing 11. Preferably battery 16 is located within the housing 11 to enable removal—either to be recharged or replaced. For example, battery 16 may be accessible through access door 43 and may be interchangeable with one or more similar batteries, such that as one battery charges the other powers the dispenser 10. Battery 16 may also be charged in place by various means such as induction charging or connecting an AC power charger via a connector (not shown) or with optional AC power 17. It will be appreciated that in the case of an AC connection that electrical codes may require distance, ground fault requirements and/or stepping down and converting the voltage to DC.

### Controls 15

Controls 15 enable the heater 13 and germicidal lamp 14. Providing input to controls 15 is motion sensor 20, heater on/off switch 18 and germicidal lamp on/off switch 19. It will be appreciated that the external buttons 42 (e.g., buttons or other switches providing user input control) may be preferably located on the exterior of the housing 11 for operation by the user. However, a remote-control panel (not shown) may be utilized. In addition, indicator lamps 41 are provided to assist the user with operation of the dispenser 10.

Motion sensor 20 may operate with a wide range so that a user approaching the dispenser 10 turns on the heater 16 and germicidal lamp 14 for a predetermined period of time and/or based on time preferences. Alternatively, the motion sensor 20 may have a limited range that requires waving a hand directly in front of the sensor 20. The latter embodiment may be preferred in higher volume settings. Further, the external buttons 42 may be utilized by a user to override the motion sensor or to otherwise turn on or turn off the various components.

In some embodiments, preference levels may be set for the various components. For example, the temperature of the heated toilet paper 12 may be adjusted, the components that are turned on by the motion sensor, and/or the amount of time that the components remain turned on may be adjusted. The preference levels may be set by a programmable chip included in the controls 15 or may be set through use of an app on a smart device (e.g., a smart phone). In some embodiments the controls 15 may include a smart device and be connected to an internet, intranet, or close proximity communication (e.g., such as Bluetooth). Further, the operation of the various components, including the charge level of the battery 16, may be monitored as well as other statistics. Accordingly, the controls 15 may comprise a microcontroller and/or microprocessor for the ability to operate and regulate the components within the housing, as well as provide alerts, shut down operations due to malfunction, among others.

### Housing 11

Housing 11 provides the structure for the dispenser 10 and houses the various components. As described above, a preferred embodiment housing 11 includes an opening on the bottom through which the toilet paper 12 is dispensed. In operation, the user may grasp the free end of the outermost layer of toilet paper to rotate and dispense the toilet paper through the open bottom. Alternatively, air may be used to rotate the toilet paper or a motor activated by a second motion sensor or “dispense” button (not shown) may be used to engage and rotate the spindle on which the toilet paper 12 is mounted.

As shown in FIG. 2, in some embodiments the housing 11′ is located above the toilet paper 12.

A preferred embodiment housing 11 is constructed of metal and may include a variety of standard fixture finishes. The preferred embodiment can also be made from a variety of materials including but not limited to plastics, wood, glass, mixed materials, ceramics, and recyclable materials, among others. In the case of metal, finishes may include copper, chrome, polished brass, satin brass, oil-rubbed bronze, matte black, gold, brushed nickel, polished nickel, and stainless steel. Preferably, the design of the housing 11 is functional and may support add-on components. For example, a shelf top, a Bluetooth charger for smart devices and phones, attachable drawers, mirrors, and stands may be included.

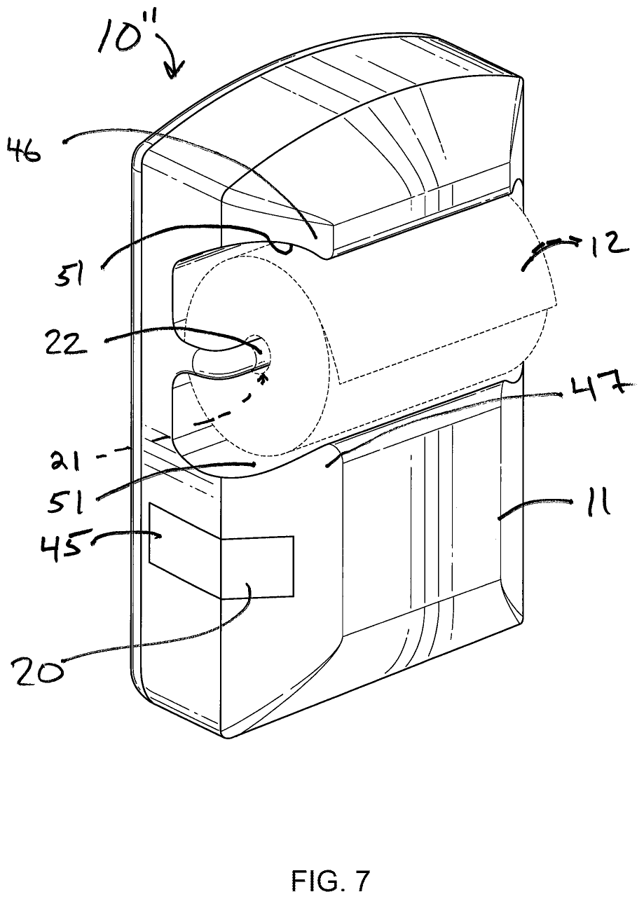

Next referring to FIG. 7, there is illustrated an embodiment including a dispenser 10″ wherein the tissue paper 12 roll is mounted on the front surface of the housing 11. The toilet paper 12 roll includes a tube 21 mounted rotationally on rod 22. Motion sensor 20 is located in this embodiment on a front surface of the housing 11. A designated area 45 provides a location for the operational lights 41 and buttons 42. However, it will be appreciated that the designated surface 45 may comprise a touch screen controlling the operation of the dispenser.

Upper arcuate projection 46 and lower arcuate projection 47 include surfaces 51 facing the toilet paper 12 roll. The facing surfaces 51 generally curve proximately above and below the toilet paper 12 roll without interfering with the rotation of the roll during dispensing. The facing surfaces 51 of the arcuate projections 46, 47 are thereby arranged to assist the warmed air (e.g., from the heater 13) to circulate about the outer surface of the toilet paper 12 roll. Louvers and/or slots provide for circulation from the heater 13 and the UV light from germicidal lamp 14 located inside the housing 11.

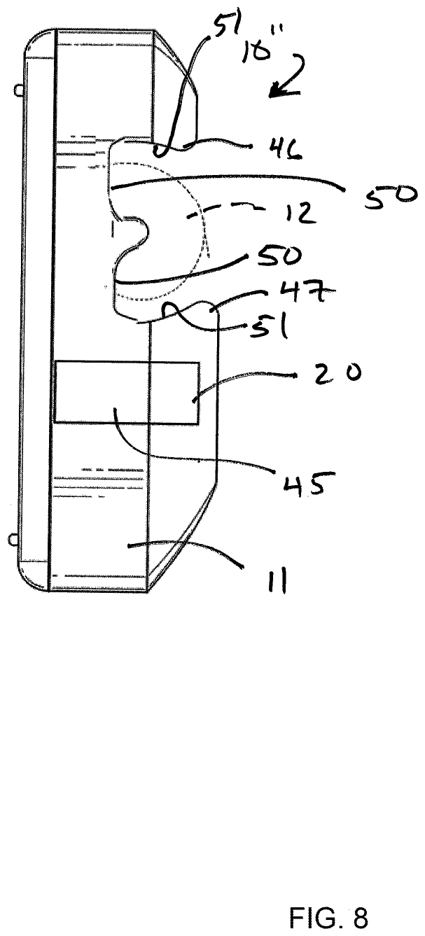

In FIG. 8, an alternative embodiment of the dispenser 10″ described in connection with FIG. 7 is illustrated. The components are generally similar. However, the side edges 50 of the housing 11 in the area occupied by the toilet paper 12 roll extend further from the rear of the housing 11 toward the front of the housing 11. This results in a more enclosed area about the toilet paper 12 roll—with the result that the toilet paper 12 roll may be warmed more efficiently. In particular, this arrangement may be utilized when the heater 13 is a polyimide heater film (described above). As noted above, such film may include an adhesive on one side such that the heater may be arranged and configured to be placed on the facing surfaces 51.

### Smart Device 202

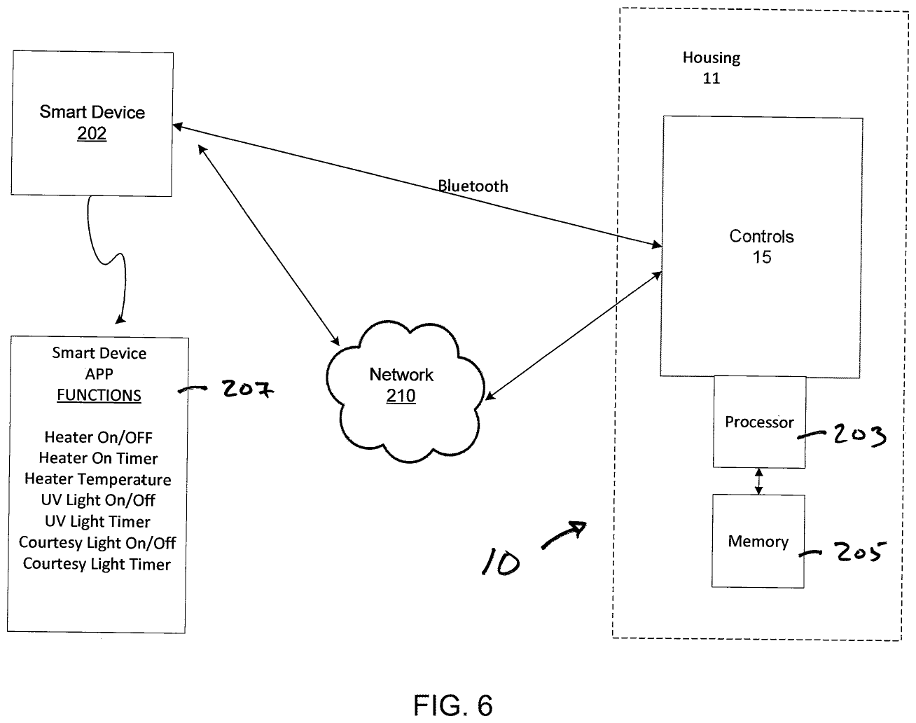

Turning to FIG. 6, a schematic of a smart device 202 which may be utilized to operate and control various parameters of the dispenser 10 is illustrated. The smart device 202 is in communication with the controls 15 via a communication network 210. The communication network 104 may include any type of wireless network, a wired network, or any communication network known in the art. For example, wireless connections can include cellular network connections and connections made using local internet protocol. Alternatively, short distance connection protocols such as Bluetooth connections may be utilized.

In the example shown in FIG. 6, a processor 203 and a memory 205 are provided for enabling features and components of the dispenser 10. The memory 205 may include a random-access memory (“RAM”) and/or a read-only memory (“ROM”). A basic input/output system that contains the basic routines that help to transfer information between the processor 203 and the controls 15 may be stored in the ROM.

It will be appreciated that the memory 205 may comprise computer-readable storage media includes volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 350.

The processor 203 may include an input/output controller for receiving and processing input from a number of other devices, including a touch user interface display screen, or another type of input device. Similarly, the input/output controller may provide output to enable and operate the controls 15 and may also provide output to a touch user interface display screen or other type of output device, as well.

As mentioned briefly above, the memory 205 can store software instructions and data. The software instructions include an operating system suitable for controlling the operation of the controls 15. Thus, the stored software instructions, that when executed by the processor 203 cause the controls 15 to provide the functionality discussed in this document.

The smart device 202 may be a smartphone, tablet, computer or other processor enabled device. Preferably the smart device 202 includes instructions or application that provides a user with options to enable, control and specify user options for the dispenser 10. For example, as shown in FIG. 6, representative options for a user of the smart device 202 may include one or more of the following functions: heater 13 on/off; heater 13 on time; heater 13 temperature; germicidal lamp 14 (e.g., UV light in FIG. 6) on/off; germicidal light 14 timer; courtesy light on/off; and/or courtesy light timer. In this manner, user preferences may be selected in connection with the dispenser 10.

### Representative Circuits

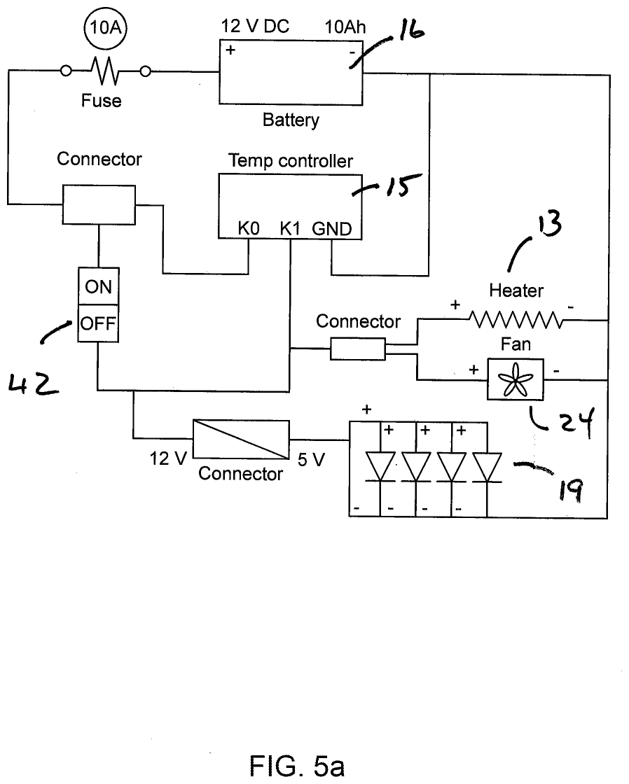

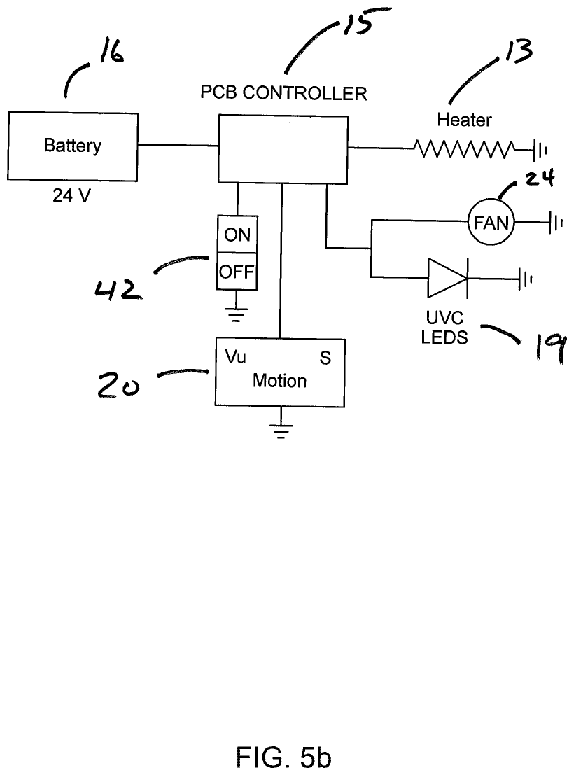

Turning now to FIGS. 5a and 5b, representative circuits and components are illustrated for the various electrical elements of dispenser 10. FIG. 5a illustrates the components associated with a 12 volt DC embodiment, while FIG. 5b illustrates the components associated with a 24 volt DC embodiment. Due to the similarity of the components, FIG. 5a will be discussed in detail. However, it will be appreciated that the described components similarly apply to FIG. 5b with the controller 15, battery 16 size, and motion sensor 20 being arranged and configured for that circuit.

In FIG. 5a battery 16 is a 12 volt battery (in the representative circuit having a 10 Ah capacity). The positive terminal of the battery 16 may be connected through an appropriately sized fuse to controls 15. In this representative circuit, the controls 15 comprises a temperature controller. The battery 16 is further connected to on/off switch 42. In the on position, the on/off switch 42 provides a voltage to the controls 15, the heater 13 and the fan 24 (the latter two via a suitable connector). In addition, the germicidal lights 19 receive power. In the representative circuit the germicidal lights comprise LED lights. In the latter case, the voltage may be stepped down to 5 volts (or other operating voltage) for the germicidal lights 19. As is well known in the art, the various devices are then appropriately connected to the negative terminal of the battery 16.

### Example Embodiment

Using a lithium-ion battery an on/off switch is connected. Also connected is a heat-whisper fan, UVC LEDs, LEDs, a thermal temperature controller together to make a master positive terminal connection, and connecting into the on/off switch. It will be appreciated that necessary negative/ground connections are made to a bus or negative terminal on the battery.

More specifically, in an example embodiment, a rechargeable ExpertPower LiFePO4 Lithium Iron Phosphate EP1210 Deep Cycle Battery (10 Ah, 12.8V and 128 Wh) may be connected using primary wire to both the positive and negative terminals. Wire may be Southwire Primary Wire 14-19 Black and Red. The Primary Wire from the positive terminal of the battery may include a Bussmann ATC 10 Amp Fuse in-line. An On/Off Switch Relay is connected electrically between the fuse and an Fdit Air Heater Fan for Small Room Space (12 v 100 W)—which includes the fan and heating element. The On/Off Switch Relay also connects to UVC LEDs. For the UVC LEDs, a resistor or converter may be used for the specific UVC LEDs.

A Temperature Controller (e.g., a RioRand 12V DC Digital Cooling/Heating Thermostat) may be used to provide temperature control. The Temperature Controller may be used in connection with a 10 A Relay with Waterproof Sensor Probe.

Using a lithium-ion battery, connect an on/off switch. From there, connect a heat-whisper fan, UVC LEDs, LEDs, a thermal temperature controller together to make a master positive terminal connection, connecting into the on/off switch.

### In Operation

Now referring to FIG. 3a, at the designation 300 there is illustrated a representative logical flow diagram which may be used in the operation of the dispenser 10. The sequence begins at block 301 and moves to block 302 where a motion sensor 20 (or on/off buttons 42 activated by a user) initiates the dispenser 10 to move into an operative mode. At block 303 energy is supplied from the battery 16 (or other power source) and at blocks 304 and 305 digital inputs and analog inputs are read and determined. Moving to block 306, based on the inputs from the previous blocks, the heat element 13, fan, and germicidal lamp 14 are turned on in accordance with the inputs.

At block 307, temperature controllers and timers are engaged to establish and maintain a time and temperature level (or other predetermined levels—which may be based on buttons 42) of the heater 13 and the germicidal light 14. Upon completion of the functions, if the motion sensor 20 or buttons 42 are engaged or reengaged, the dispenser 10 returns to block 303 to continue operation. Alternatively, the functions turn off at block 308 and the method ends at block 309.

FIG. 3b illustrates an example alternative embodiment having a logic flow 300′. The logical flow is similar to that described in connection with FIG. 3a. Accordingly, the similar steps will not be repeated. The differences begin at block 307′. Here when the temperature controllers and timers are engaged, the controllers and timers communicate with a processor to establish and maintain user preferences related to the time and temperature level (or other predetermined levels) of the heater 13 and the germicidal light 14. The preference levels may be set by programming the processor or utilizing a smart device app as described above. Upon completion of the functions, if the motion sensor 20 or buttons 42 are engaged or reengaged, the dispenser 10 returns to block 303 to continue operation.

Alternatively, the flow moves to block 310 where various safety controls are monitored to determine if any parameters have been exceeded. In this embodiment block 310 preferably communicates with the logical flow beginning at block 303 and can discontinue the power to the dispenser 10 in the event that any monitored parameter is exceeded. In this manner continuous monitoring of the dispenser 10 may be provided. If no further functions are desired, the dispenser functions turn off at block 308 and the method ends at block 309.

### ALTERNATIVE EMBODIMENTS

In one alternative embodiment, the housing 11 may include a mechanism that allows a material to be sprayed upon the toilet tissue. The spray may include a disinfectant, lotions, and hygiene cleansing solutions, among others.

In another alternative embodiment, the housing 11 may include a shelving unit. The shelving unit may include additional features such as a phone charger, a drawer for hygiene products, a compartment for wet wipes, a stand for further stock of toilet tissue, and/or a built in deodorizer

In a third embodiment, the heating may be based on conduction, such as with infrared heating, the heat targeted to the roll of toilet tissue 12.

In a fourth embodiment, a cooler is provided for cooling the toilet tissue for immediate use. Preferably the cooler is arranged and configured to select and enact different levels of cooling. In addition, the levels of cooling may be set as preferences by a user. For example, air flow may be directed from a cooling component toward the toilet tissue to enable quickly cooling the top layer(s). Accordingly, a fan may be provided to generate the air flow. A thermal shield may be provided to insulate the housing 11.

In a fifth embodiment, the fan used in connection with the heating element 13 or the cooler (not shown) may function as a dispenser of the toilet tissue by targeting the air on the rotatably mounted toilet tissue. More specifically providing air flow volume and trajectory may be used to dispense an effective amount of toilet tissue.

In any of the above embodiments, various safety features may be provided. Such safety features may take the form of fuses, ground fault circuits and/or communications to control components. The monitored parameters may be based on time, thermal controls, temperature testing, voltage, leakage current, etc.

In view of the foregoing, it will be appreciated that the principles of this invention apply to a method and apparatus for dispensing toilet paper. While particular embodiments of the invention have been described with respect to its application, it will be understood by those skilled in the art that the invention is not limited by such application or embodiment or the particular components disclosed and described herein.

Accordingly, other components that embody the principles of this invention can be configured within the spirit and intent of this invention. The arrangement described herein is provided as only one example of an embodiment that incorporates and practices the principles of this invention. Other modifications and alterations are well within the knowledge of those skilled in the art and are to be included within the broad scope of the appended claims.

What is claimed is:

1. An apparatus for dispensing toilet tissue, comprising:
a) a housing;
b) a heating element mounted on or within the housing and proximate the toilet tissue;
c) a germicidal lamp located within the housing, the germicidal lamp arranged and configured to irradiate the toilet tissue; and
d) a control device, the control device operatively connected to the heating element and the germicidal lamp, the control device initiating operation of the heating element and the germicidal lamp.

2. The apparatus of claim 1, wherein the heating element is located within the housing, the toilet tissue is rotatably mounted within at least a portion of the housing and the housing includes a first void through which the toilet tissue is dispensed.

3. The apparatus of claim 1, wherein the housing is located above an existing toilet tissue holder, the housing including one or more second voids within a bottom portion of the housing, the heating element is located within the housing, and wherein in operation a fan directs heated air from the heating element, through the voids and onto the toilet tissue.

4. The apparatus of claim 1, wherein the control device includes on/off switches and a timer for the heating element.

5. The apparatus of claim 1, wherein the control device includes a timer for the germicidal lamp.

6. The apparatus of claim 1, wherein the control device includes a microprocessor and memory, the microprocessor being programmable to individual user preferences on duration and temperature of the toilet tissue heating and to store the individual preferences in the memory location.

7. The apparatus of claim 1, further comprising a motion sensor, the motion sensor initiating operation operatively connected to the control device, and wherein the motion sensor is arranged and configured to initiate at least one of the heating element and the germicidal lamp.

8. The apparatus of claim 7, wherein the motion sensor is located on the top of the housing, whereby a user motion above the housing enables the motion sensor.

9. The apparatus of claim 7, wherein the motion sensor is located on the bottom of the housing, whereby a user motion below the housing enables the motion sensor.

10. The apparatus of claim 6, further comprising a courtesy light.

11. The apparatus of claim 10, wherein the control device is arranged and configured to communicate with a smart device and to receive user selected preferences from the smart device related to at least one of the heating element, the germicidal lamp, and the courtesy light.

12. The apparatus of claim 1, wherein the heating element is a polyimide heater film.

13. The apparatus of claim 12, wherein the heating element is mounted on an external surface of the housing.

14. A method of dispensing from a roll of toilet tissue in an ergonomic manner, comprising:
a) transmitting heat to at least the outer layers of the roll of toilet tissue from a heating element located on or within a housing;
b) shining a germicidal lamp on the outer layers of the roll of toilet tissue from a light source located within the housing; and
c) providing a control device, the control device operatively connected to the heating element and the germicidal lamp and arranged and configured for selectively operation of the heating element and the germicidal lamp.

15. The method of claim 14, further comprising rotatably mounting the roll of toilet tissue in the housing and providing a first void in the housing through which the toilet tissue is dispensed.

16. The method of claim 14, further comprising locating the housing above an existing toilet tissue holder, forming one or more second voids within a bottom portion of the housing, and directing heated air through the second voids onto the toilet tissue.

17. The method of claim 14, further comprising operatively connecting user preference controls to the control device to provide user preferred timing and level of heat from the heating element, user preferred timing for the germicidal lamp, and user defined operation and timing of a courtesy light.

18. The method of claim 17, wherein the control device includes a microprocessor and memory, the microprocessor being programmable to individual user preferences on duration and temperature of the toilet tissue heating and to store the individual preferences in the memory location.

19. The method of apparatus of claim 18, wherein the control device is arranged and configured to communicate with a smart device and to receive user selected preferences from the smart device related to at least one of the heating element, the germicidal lamp, and the courtesy light.

20. The method of claim 14, further comprising sensing the motion of a user with a motion sensor operatively connected to the control switches, the motion sensor arranged and configured to initiate at least one of the heating element and the germicidal lamp.
